(19) United States
(12) Patent Application Publication
(10) Pub. No.: US 2026/0266812 A1
(43) Pub. Date: Sep. 10, 2026

(54) METHOD FOR DIRECTLY DETECTING THE PRESENCE OF A TEST SUBSTANCE

(71) Applicants: Constantin ODEFEY, Hamburg (DE); Olaf STILLER, Krefeld (DE)

(72) Inventor: Constantin ODEFEY, Hamburg (DE)

(21) Appl. No.: 19/158,024

(22) PCT Filed: Feb. 20, 2023
(86) PCT No.: PCT/EP2023/054157

§ 371 Date: Aug. 19, 2025

(51) Int. Cl. G01N 33/543; G01N 21/78; G01N 33/569; G01N 33/58

(52) U.S. Cl. CPC G01N 33/54326; G01N 21/78; G01N 33/54346; G01N 33/56911; G01N 33/56983; G01N 33/582; G01N 33/585

(57) ABSTRACT

The invention relates to a method comprising the following steps: providing a test fluid in which a test substance particle is present in a first case; providing a first nanobead fluid containing a first magnetic nanobead, a second nanobead fluid containing a second magnetic nanobead, a third nanobead fluid containing a non-magnetic nanobead, and a marker fluid containing a marker particle; creating a second fluid by mixing the test fluid with the first nanobead fluid and mixing at least the first magnetic nanobead of the resulting fluid with the third nanobead fluid, whereupon the resulting fluid is referred to as the second fluid; removing the first magnetic nanobead from the second fluid by means of a first magnetic force; creating a third fluid by mixing the second nanobead fluid and the marker fluid into the second fluid; and removing the second magnetic nanobead from the third fluid by means of a second magnetic force, wherein, in the first case, the marker particle remains in the third fluid.

### CROSS-REFERENCE TO RELATED APPLICATIONS

This application is a US National Stage of PCT/EP2023/054157 filed on Feb. 20, 2023, which is incorporated herein by reference.

### FIELD

The present invention relates to a method for directly detecting the presence of a test substance in a test fluid.

### BACKGROUND

Methods for detecting the presence of test substances in a test fluid are used in a variety of ways in chemistry, (micro)biology, medical analysis, and biochemistry. A typical test substance is a pathogenic virus or bacterium that originates from a corresponding patient smear and that was mixed into liquid. The detection of poisonous substances in a test fluid is also a typical field of application of such methods. The fluids may, in particular, be homogenous (single-phase) liquids, consisting of a single liquid component or of a homogenous mixture of multiple liquid components (solutions). Furthermore, fluids may be heterogenous (multi-phase) mixtures of a liquid with another liquid (emulsions) or a solid (suspensions).

In order to detect the presence of a test substance, marker particles such as dyes are typically used, which alter a physical property of the test fluid depending on the presence of the test substance. To this end, the marker particle is added to the test fluid. Subsequently, the physical properties of the test fluid, e.g. its color or electrical conductivity, are altered. The marker particle is designed such that it directly or indirectly binds to a test substance particle. After the marker particle is added, the test substance particle and the marker particle bound thereto are removed from the test fluid using suitable measures. In the event that no test substance was present in the test fluid, the marker particle remains in the test fluid. The physical properties of the test fluid are then altered accordingly by the marker particle and this alteration must then merely be recorded, for example by analyzing the color of the test fluid.

In order to remove the test substance particle from the test fluid, nanobeads can be used, for example. Nanobeads are small particles of a wide variety of shapes which have a functionalized surface. The functionalized surface can be provided with suitable binding sites depending on the test substances to be detected. The binding sites form a binding with the complementary binding sites of the test substance particle when a nanobead and the test substance particle come together. This may, for example, be an antigen-antibody binding. Furthermore, nanobeads typically contain iron and are thus magnetic. By applying a magnetic force, nanobeads and test substance particles bound thereto can be removed from the test fluid.

One disadvantage is that, when testing a test fluid for the presence of the test substance particle, the marker particle does not remain in the test fluid if the test substance particle is present. It is only when no test substance particle is present in the test fluid that the marker particle remains in the test fluid and the test shows a positive result. It is therefore a reciprocal test, in which a positive test result indicates the absence of the test substance particle.

EP 3 147 028 A1 discloses a method for magnetically separating nanobeads from a first fluid, which method comprises the following steps: providing the first fluid with the nanobeads located therein; introducing a sleeve body into the first fluid, wherein a first magnet is arranged in the sleeve body and can be shifted along a longitudinal axis of the sleeve body; collecting nanobeads at the sleeve wall by means of the force effect of a first magnetic field of the first magnet; removing the sleeve body with the collected nanobeads from the first fluid; introducing the sleeve body with the collected nanobeads into a second fluid; and providing a magnetic field by means of a device providing a second magnetic field, the magnetic pole orientation of which magnetic field is opposite the magnetic pole orientation of the first magnet in the sleeve body, such that the first magnet in the sleeve body is repelled by the magnetic field and is shifted in the sleeve body, wherein the device providing a second magnetic field and the sleeve body do not overlap in a direction perpendicular to the longitudinal axis.

### SUMMARY

Proceeding from this, the object of the invention is to provide a nanobead-based test method that yields a positive response in the presence of a test substance in a test fluid.

This object is solved by a method according to claim 1. Advantageous embodiments are given in the dependent claims and in the following description.

The method according to the invention for detecting a test substance in a test fluid comprises the following steps:
i. providing a first fluid, the test fluid, in which a test substance particle is present in a first case and the test substance particle is not present in a second case,
   wherein the test substance particle has binding sites of a first type,
ii. providing
   a first nanobead fluid containing a first magnetic nanobead with a complementary binding site of a first type,
   a second nanobead fluid containing a second magnetic nanobead with a binding site of a second type,
   a third nanobead fluid containing a non-magnetic nanobead with a complementary binding site of a first type, a complementary binding site of a second type, and a complementary binding site of a third type,
   a marker fluid containing a marker particle with a binding site of a third type,
   wherein, in the presence of corresponding binding partners in the same fluid,
      a binding site of a first type of a test substance particle forms a binding of a first type with a complementary binding site of a first type of a magnetic nanobead of a first type and of a non-magnetic nanobead,
      a binding site of a second type of a magnetic nanobead of a second type forms a binding of a second type with a complementary binding site of a second type of a non-magnetic nanobead,
      a binding site of a third type of a marker particle forms a binding of a third type with a complementary binding site of a third type of a non-magnetic nanobead,
iii. creating a second fluid
   by mixing the test fluid with the first nanobead fluid and mixing at least the first magnetic nanobead of the resulting fluid with the third nanobead fluid, whereupon the resulting fluid is referred to as the second fluid,
iv. removing the first magnetic nanobead from the second fluid by means of a first magnetic force,
   wherein, in the first case, the test substance particle bound to the first magnetic nanobead and the non-magnetic nanobead bound to the test substance particle are also removed from the second fluid
   or, in the second case, the non-magnetic nanobead remains in the second fluid,
v. creating a third fluid
   by mixing the second nanobead fluid and the marker fluid into the second fluid, whereupon the mixture is referred to as the third fluid,
vi. removing the second magnetic nanobead from the third fluid by means of a second magnetic force,
   wherein, in the first case, due to the absence of the non-magnetic nanobead in the third fluid, the marker particle is not indirectly bound to the second magnetic nanobead, and therefore it remains in the third fluid after the second magnetic nanobead is removed, or
   wherein, in the second case, the non-magnetic nanobead is bound to the second magnetic nanobead and the marker particle is bound to the non-magnetic nanobead, and therefore the marker particle is removed from the third fluid together with the second magnetic nanobead and the non-magnetic marker particle.

The method according to the invention is characterized by the use of the non-magnetic nanobead and two different magnetic nanobeads. The non-magnetic nanobead makes it possible, when the method is applied, for the presence of the marker particle in the final, third fluid to directly indicate that the test substance particle was present in the test fluid at the start. In this way, a test based on the method according to the invention for detecting the presence of the test substance in the test fluid yields a positive result if the test substance particle was present in the test fluid.

The first magnetic nanobead serves to remove the non-magnetic nanobead from the second fluid in the presence of the test substance particle. This happens after the second fluid is created, which is produced by mixing at least the test fluid and first nanobead fluid and by mixing the first magnetic nanobead of the resulting mixture with the third nanobead fluid. This binds the first magnetic nanobead to the test substance particle when the first nanobead fluid and test fluid are mixed, provided that the test substance particle is contained in the test fluid, and the test substance particle, which is if applicable bound to the first magnetically bound nanobead, binds to the non-magnetic nanobead when the first magnetic nanobead is introduced into the third nanobead fluid. Subsequently, the first magnetic nanobead is removed from the second fluid by means of the first magnetic force. If the test substance particle is not present, the non-magnetic nanobead remains in the second fluid after the first magnetic nanobead has been removed from the second fluid, because it cannot bind to the first magnetic nanobead directly, but rather only indirectly via the test substance particle.

In the present application, “mixing,” “mixing in,” and “admixing” (in the following collectively “mixing”) of fluids should be understood to mean that fluids are brought together such that they are in direct contact with one another. The mixing of fluids can, in particular, take place by pouring fluids together or pouring them one on top of the other or by introducing one fluid into another fluid. A fluid can be introduced, for example, by adding a fluid by means of a pipette into a fluid standing ready in a vessel. Preferably, the mixing of fluids takes place such that they are evenly distributed in one another, for example by stirring or shaking them. However, mixing also includes bringing together fluids which are unevenly distributed over the total volume occupied by the fluids. The crucial factor is that the fluids come into contact with one another, such that particles contained in the various fluids (e.g. test substance particles, magnetic nanobeads, non-magnetic nanobeads, or marker particles) interact with one another and can form bindings with one another, provided that they are binding partners within the context of the invention.

The second magnetic nanobead serves to remove the marker particle from the third fluid if the test substance particle was originally absent. This removal takes place after the third fluid is formed from the second fluid by mixing the marker particle and the second magnetic nanobead into the second fluid. Similarly to before with the second fluid, the second magnetic force is used to remove the second magnetic nanobead from the third fluid. The marker particle cannot bind to the second magnetic nanobead directly, but rather only indirectly via the non-magnetic nanobead, which can bind both to the second magnetic nanobead and to the marker particle. However, the non-magnetic nanobead is only present in the third fluid if it was not previously removed from the second fluid, which is only the case if it could bind indirectly via the test substance particle to the first magnetic nanobead and be removed therewith from the second fluid. The marker particle is thus only removed from the third fluid if the non-magnetic nanobead was present in the third fluid, which, in turn, is only the case if the test substance particle was not originally present in the test substance fluid.

The presence of the test substance particle is thus the deciding factor in whether the non-magnetic nanobead is absent in the third fluid. It is only in the absence of the non-magnetic nanobead in the third fluid, which corresponds to the presence of the test substance particle in the second fluid, that the marker particle remains in the third fluid after the second magnetic nanobead has been removed. The test is therefore also referred to as reciprocal-reversible.

The advantage of the method according to the invention is that the marker remaining in the third fluid, i.e. a positive test result, directly indicates the original presence of the test substance particle in the test fluid. This produces advantages when handling the method during use in a laboratory.

A test based on the method does not yield a result if one of the particles used has a defect that renders the test unusable. When the test yields a result, a laboratory can thus rely on the fact that a test substance particle is also actually present and that there is not just a false positive result of the test.

Furthermore, a test based on the method is particularly suitable for pooling studies. In studies of this kind, it is checked whether multiple different potential test substances (e.g. bacteria or viruses) are found in the test fluid at the same time. The method according to the invention makes it possible to directly indicate the presence of a sought, specific test substance instead of merely indirectly excluding the fact that none of the potential test substances were present in the test fluid.

Moreover, it is advantageous that the non-magnetic nanobeads are used to bind the marker particles. The non-magnetic nanobeads may therefore be designed to enter into a binding with a very large number of marker particles at the same time. In this way, a test based on the method may be highly sensitive. Few non-magnetic nanobeads are sufficient for removing a large quantity of marker particles and thus for generating a test signal that is easy to detect. If even a single non-magnetic nanobead is removed from the second fluid due to the presence of a single test substance particle, a significantly greater number of marker particles remain in the test fluid than in a case where no test substance particle is present and all non-magnetic nanobeads remain in the third fluid for binding marker particles.

According to one embodiment of the method, the third nanobead fluid is mixed with the test fluid and with the first nanobead fluid in order to create the second fluid.

When performing the method, there are alternatives for creating the second fluid. In one alternative, the first nanobead fluid, which contains the first magnetic nanobeads, and the test fluid are mixed directly with the third nanobead fluid, which contains the non-magnetic nanobeads. Such a way of performing the method is particularly simple.

According to one embodiment of the method, in order to create the second fluid,
firstly, the first nanobead fluid is mixed with the test fluid,
secondly, the first magnetic nanobead is removed from the test fluid by means of a third magnetic force, and
thirdly, the first magnetic nanobead removed from the test fluid is introduced into the third nanobead fluid by means of a fourth magnetic force.

In addition to the above-described alternative for creating the second fluid, it is also possible to first mix only the first nanobead fluid with the test fluid and then to remove the first magnetic nanobead from this mixture again using the first magnetic force. If a test substance particle is present, it is then removed from the original test fluid together with the first magnetic nanobead. The first nanobead and the test substance particle, which is if applicable bound thereto, are then introduced into the third nanobead fluid and only then come into contact with the non-magnetic nanobead. It is advantageous here that there is no risk that the non-magnetic nanobead will react with impurities in the test fluid. Moreover, the vessel for the third nanobead fluid can be small, such that the concentration of the test substance particles in this fluid is greater than in the original test fluid. In this way, the sensitivity of a test based on the method in this alternative is increased.

According to one embodiment of the method,
the test substance particle has two or more binding sites of a first type,
the first magnetic nanobead has a single or more complementary binding sites of a first type,
the second magnetic nanobead has a single or more binding sites of a second type,
the non-magnetic nanobead has a single or more complementary binding sites of a first type, a single or more complementary binding sites of a second type, a single or more complementary binding sites of a third type, and
the marker particle has a single or more binding sites of a third type.

Typically, the particles involved have multiple binding sites or else complementary binding sites. If the particles only have one binding site, production is complicated. In particular, the presence of multiple complementary binding sites of a third type with just one complementary binding site of a first type on the non-magnetic nanobead ensures a favorable ratio of the number of test substance particles to a change in the concentration of the marker particles in the final fluid. This favorable ratio means that a test based on the method is very sensitive.

According to one embodiment of the method,
the test fluid contains a single or multiple test substance particles and/or
the first nanobead fluid contains a single or multiple first magnetic nanobeads and/or
the second nanobead fluid contains a single or multiple second magnetic nanobeads and/or
the third nanobead fluid contains a single or multiple non-magnetic nanobeads and/or
the first marker fluid contains a single or multiple marker particles.

It is typical to use a plurality of all particles used. This increases the probability that a binding will form between the respective particles. However, it is also possible, if only one test substance particle is present, to use only one other of the other particles in each case for detecting this single test substance particle.

In a further embodiment, the method is used to detect the test substance in the test fluid if the number of test substance particles is within the femto- and/or atto- and/or zeptomolar range. In a further embodiment, the method is used for single-molecule counting. An application with a larger number of test substance particles is also possible.

According to one embodiment of the method, the binding of a first type is an antibody-antigen binding or an oligonucleotide binding or a lipid binding or a glucose binding.

The nanobeads can be produced with various binding sites or else complementary binding sites. As a result, many different test substances that have different types of binding sites of a first type can be investigated using the method according to the invention. In the case of many test substances, for example viruses or bacteria, a binding of a first type is formed as an antibody-antigen binding between the test substance particle and first magnetic nanobead or else non-magnetic nanobead, but with other test substances, for example poisonous substances, there may be an oligonucleotide binding, lipid binding, or glucose binding.

According to one embodiment of the method, the binding of a second type is an antibody-antigen binding or an oligonucleotide binding or a lipid binding or a glucose binding.

In the case of the binding of a second type as well, different variants can be implemented. The manner in which the second magnetic nanobead and the non-magnetic nanobead are equipped during production depends on the external conditions to which the method is to be exposed. It is therefore made more flexible by the possibility of using various types of bindings of a second type.

According to one embodiment of the method, the binding of a third type is an antibody-antigen binding or an oligonucleotide binding or a lipid binding or a glucose binding.

By using various bindings of a third type, a large number of possible marker particles that have different types of binding sites of a third type can be used for the method. In this way, the method is tailored to specific circumstances in a laboratory in terms of the analytics present for detecting the marker particle.

According to one embodiment of the method, the marker particle is a colored particle, a luminous particle, a fluorescent particle, a particle that influences the electrical conductivity of a liquid, or a radioactive particle.

Different marker particles can be used depending on the available analytics. Dyes that color the third fluid and that are recognizable to the naked eye are typical. These dyes may glow or fluoresce, which means that they glow when irradiated by a light source, to improve recognizability. Especially fluorescent dyes can be detected even at a very low concentration. Even a change in the concentration is easily discernible. Furthermore, a change in the electrical conductivity of a fluid can be precisely detected depending on the concentration of the corresponding marker particles contained in the fluid. To this end, distinctive marker particles are consequently very well suited for the method as well. Similarly, radioactive marker particles can be detected accurately and are thus suitable as marker particles.

According to one embodiment of the method, an additional test substance particle with an alternative binding site of a first type is located in the test fluid,
an additional first nanobead fluid is provided which contains an alternative first nanobead with an alternative complementary binding site of a first type, and
an additional third nanobead fluid is provided which contains an alternative non-magnetic nanobead with an alternative complementary binding site of a first type and an alternative complementary binding site of a third type, and
an additional marker fluid is provided which contains an alternative marker particle with an alternative binding site of a third type,
wherein the additional first nanobead fluid, the additional third nanobead fluid, and the additional marker fluid are used in the method in the same steps and in the same way as the first nanobead fluid, the third nanobead fluid, and the marker fluid, respectively.

A pooling test, in which the presence of various types of test substances is investigated at the same time, can advantageously be performed with nanobeads that are tailored to the respective test substances. To this end, alternative nanobeads with alternative binding sites are used which are suitable for detecting an additional test substance that is different from the original test substance. The method is carried out in the same way as described above when alternative nanobeads are used, but multiple nanobead fluids are used at the same time in the respective method steps. In particular, an alternative marker fluid that contains a marker particle that only binds to the alternative, non-magnetic nanobeads is used. Said non-magnetic nanobead is, in turn, only capable of forming a binding with a test substance particle of a particular type. As a result, the presence of a test substance of a specific type is also indicated by means of a corresponding marker particle. Only the respectively corresponding marker particles remain in the final fluid depending on the presence of the respective, different test substance types, such that, for example, a characteristic coloring is produced in the third fluid that makes it possible to conclude which type of test substances were originally present and which were not present.

According to one embodiment of the method, the test fluid contains a single or multiple additional test substance particles and/or
the first additional nanobead fluid contains a single or multiple alternative first nanobeads and/or
the third additional nanobead fluid contains a single or multiple alternative third nanobeads and/or
the additional marker fluid contains a single or multiple alternative marker particles.

As in the case of the non-alternative test substance particles, nanobeads, and marker particles, a plurality of the alternative test substance particles, nanobeads, and marker particles may also be provided in each case.

According to one embodiment of the method,
the first magnetic force is applied by means of a first magnet and/or
the second magnetic force is applied by means of a second magnet and/or
wherein
   the relevant magnet is immersed in the relevant fluid,
   as a result of magnetic forces, the relevant magnetic nanobead moves toward the relevant magnet and is then held on the relevant magnet,
   such that the magnet and nanobead are removed together from the fluid in order to remove the nanobead.

Achieving the above-mentioned magnetic forces using a magnet that is immersed in the relevant fluid has the advantage that the respective nanobeads are held directly on the relevant magnet. The magnet can then be easily removed from the relevant fluid in order to remove the nanobeads from the fluid. The nanobeads and test substance particles bound thereto or indirectly bound marker particles are thus also easily removed from the fluid. By moving the magnet through the relevant fluid after it has been immersed therein, it can be ensured that all nanobeads actually come into contact with the magnet. This ensures that, as far as possible, all nanobeads that are potentially to be removed are removed from the fluid. Furthermore, the nanobeads are then held on the magnet, which is free from impurities. This increases the reliability of the method.

According to one embodiment of the method, when creating the second fluid,
the third magnetic force is applied by means of a third magnet
   which is immersed in the test fluid,
   as a result of magnetic forces, the first magnetic nanobead moves toward the third magnet and is then held on the third magnet,
   such that the magnet and first magnetic nanobead are removed together from the fluid in order to remove the first magnetic nanobead, and
the fourth magnetic force is generated by reversing the polarity of the third magnet, such that the first magnetic nanobead held on the magnet is repelled when the second fluid is created, wherein, in order to introduce the nanobead into the third nanobead fluid, the first magnet is first immersed in the third nanobead fluid before the polarity of the magnet is reversed.

In an alternative embodiment of the method in which the second fluid is created in multiple substeps, the first magnetic nanobead must first be removed from the test fluid using a third magnetic force before it is then introduced into the third nanobead fluid in order to create the second test fluid. For said introduction, a fourth magnetic force is used. It is advantageous if the fourth magnetic force is caused by a polarity reversal of the third magnet, for which purpose said third magnet is designed as an electromagnet, for example. The first magnetic nanobead is held on the magnet after same has been held in the mixture of the test fluid and first nanobead fluid. As soon as the third magnet has been removed from the mixture and held in the third nanobead, its polarity can be reversed, such that the magnetic nanobeads are repelled. Then, no further magnets or aids are required to introduce the first magnetic nanobead into the third nanobead fluid. The third magnet must merely be held in the third nanobead fluid and its polarity reversed. In this way, the method is easy to implement.

According to one embodiment of the method, when the second fluid is created,
the third magnetic force is applied by means of a third magnet
   which is immersed in the test fluid,
   as a result of magnetic forces, the first magnetic nanobead moves toward the third magnet and is then held on the third magnet,
   such that the magnet and first magnetic nanobead are removed together from the fluid in order to remove the first magnetic nanobead, and
the fourth magnetic force is generated by means of a magnetic field impressed on the third nanobead fluid from outside, wherein the fourth magnetic force is only impressed after the third magnet, with the first magnetic nanobead held on the third magnet, has been immersed in the third nanobead fluid.

Instead of reversing the polarity of the third magnet as described above, it is also possible to generate the fourth magnetic force by means of a magnetic field impressed on the third nanobead fluid from outside. To this end, the third magnet is immersed in the third nanobead fluid with the first magnetic nanobead held thereon. Subsequently, the fourth magnetic force is generated, for example by bringing another, stronger magnet into the vicinity of the third nanobead fluid. This magnetic force is so strong that it detaches the first magnetic nanobead from the third magnet and said first magnetic nanobead, including any test substance particle potentially bound thereto, is thus introduced in a simple manner into the third nanobead fluid.

According to one embodiment of the method, the first and/or second and/or third magnet is surrounded by a protective layer, such that the relevant magnetic nanobead is held on the protective layer as a result of the magnetic forces.

If the magnets are surrounded by a protective layer, only the protective layer needs to be replaced between repetitions of the method, instead of the entire magnet having to be replaced. As a result, the costs for the method are reduced and automatability facilitated.

The combination of this embodiment with the above-described embodiment, in which a fourth magnetic force is used, is advantageous. If the third magnet can move in the protective layer surrounding it and is oriented accordingly relative to the magnetic field, it is repelled by the fourth magnetic force and is moved in the protective layer. In this way, the magnetic force of the third magnet acting on the first magnetic nanobeads can be weakened and it becomes easier to introduce the first magnetic nanobead into the third nanobead fluid.

According to one embodiment of the method, the number of test substance particles originally present in the test fluid is determined in that
a known number of non-magnetic nanobeads, in each case with a known number of complementary binding sites of a first and third type, is used,
   wherein the total number of complementary binding sites of a first type present on the non-magnetic nanobeads is greater than the number of test substance particles
   and wherein the total number of complementary binding sites of a third type present on the non-magnetic nanobeads is less than the number of marker particles, and
the difference between the marker particles remaining in the third fluid at the end and the marker particles present at the start is determined,
the number of test substance particles originally present is calculated using the difference of the marker particles with knowledge of the number of non-magnetic nanobeads.

The method can be used not only to detect the mere presence of a test substance, but with the method it is also possible to quantify the number of test substance particles present using the embodiment described above. To this end, the quantity of complementary binding sites of a first type and third type comprised by the non-magnetic nanobead must be known. This is possible by producing the nanobead accordingly. When using multiple non-magnetic nanobeads, it can be ensured that all non-magnetic nanobeads have at least approximately the same number of respective binding sites, by ensuring that all non-magnetic nanobeads are of equal size, e.g. by purifying commercially available nanobead fluids.

The complementary binding sites of a first type bind the test substance particles. If a plurality of these binding sites is present on a non-magnetic nanobead, a non-magnetic nanobead can bind multiple test substance particles. The same also applies to the marker particle, which is bound by means of complementary binding sites of a third type to the non-magnetic nanobead. The ratio of complementary binding sites of a first type to complementary binding sites of a third type on the non-magnetic nanobead indicates how many fewer marker particles remain in the third fluid when a non-magnetic nanobead has been removed from the second fluid, which in turn corresponds to a removal of the corresponding number of test substance particles. By measuring the change in the number of marker particles, with knowledge of the ratio it is possible to conclude the number of test substance particles removed from the second fluid together with non-magnetic nanobeads. Said change in the number of marker particles can be measured based on a change in the concentration of the marker particles, which is determined, for example, by the intensity of the fluorescence of the third fluid mixed with marker particles in comparison with the intensity of the fluorescence of the marker fluid with knowledge of the respective liquid quantities.

Here, it is important that there are more complementary binding sites of a first type than test substance particles and more marker particles than complementary binding sites of a third type of all non-magnetic nanobeads used. Otherwise, not all test substance particles can be bound and the corresponding number of marker particles cannot be bound to each non-magnetic nanobead, because all marker particles may already be bound even though there are still non-magnetic nanobeads present in the third fluid.

According to one embodiment of the method, the container in which the second fluid is created tapers conically downward.

The nanobeads generally have a higher density than the fluid in which they are contained. Consequently, the nanobeads collect at the bottom of the liquid without suitable mixing, and therefore all nanobeads involved in the method come together at this location. This ensures that all corresponding nanobeads can form a binding.

### BRIEF DESCRIPTION OF THE DRAWINGS

The invention is explained in more detail below based on two exemplary embodiments represented in the appended drawings, in which:

FIG. 1 a shows the test fluid in the case where the test substance particle is present;

FIG. 1 b shows the test fluid in the case where the test substance particle is absent;

FIG. 2 shows the three nanobead fluids and the marker fluid;

FIG. 3 a shows the creation of the second fluid in the case where the test substance particle is present;

FIG. 3 b shows the creation of the second fluid in the case where the test substance particle is absent;

FIG. 4 a shows the application of the first magnetic force in the case where the test substance particle is present;

FIG. 4 b shows the application of the first magnetic force in the case where the test substance particle is absent;

FIG. 5 a shows the creation of the third fluid in the case where the test substance particle is present;

FIG. 5 b shows the creation of the third fluid in the case where the test substance particle is absent;

FIG. 6 a shows the application of the second magnetic force in the case where the test substance particle is present;

FIG. 6 b shows the application of the second magnetic force in the case where the test substance particle is absent;

FIG. 7 a shows the mixing of the test fluid and of the first nanobead fluid in the case where the test substance particle is present according to an alternative embodiment;

FIG. 7 b shows the mixing of the test fluid and of the first nanobead fluid in the case where the test substance particle is absent according to an alternative embodiment;

FIG. 8 a shows the application of the first magnetic force in the case where the test substance particle is present according to an alternative embodiment;

FIG. 8 b shows the application of the first magnetic force in the case where the test substance particle is absent according to an alternative embodiment;

FIG. 9 a shows the creation of the second fluid in the case where the test substance particle is present according to an alternative embodiment;

FIG. 9 b shows the creation of the second fluid in the case where the test substance particle is absent according to an alternative embodiment.

### DETAILED DESCRIPTION

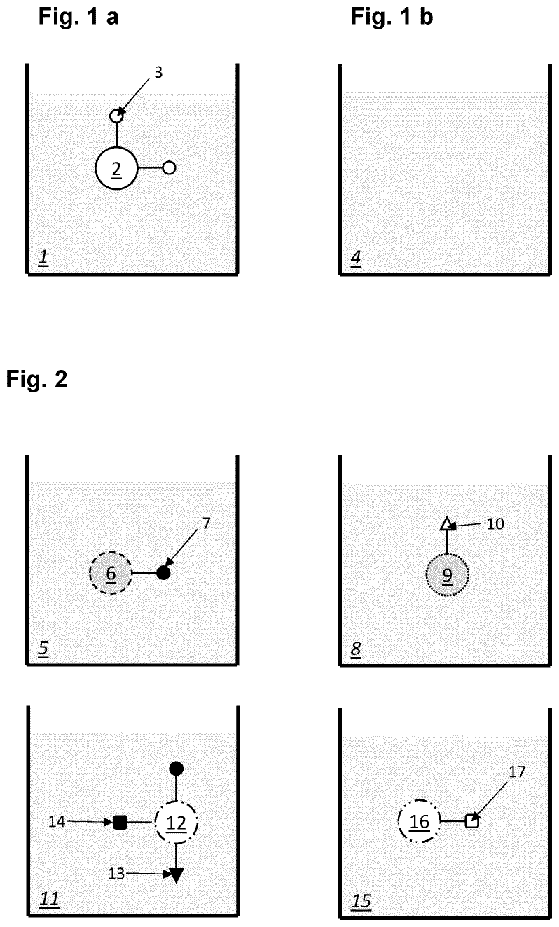

FIG. 1 a shows the test fluid 1, which is also referred to as the first fluid, in the presence of the test substance particle 2. The test substance particle 2 has a binding site of a first type 3. FIG. 1 b shows the test fluid 4 in the absence of the test substance particle.

FIG. 2 shows the first nanobead fluid 5, in which the first magnetic nanobead 6 is contained. The first magnetic nanobead 6 has a complementary binding site of a first type 7. The second nanobead fluid 8 comprises a second magnetic nanobead 9, which has a binding site of a second type 10. The third nanobead 11 comprises a non-magnetic nanobead 12, which, in addition to a complementary binding site of a first type 7, also has a complementary binding site of a second type 13 and a complementary binding site of a third type 14. The marker fluid 15 comprises a marker particle 16, which has a binding site of a third type 17.

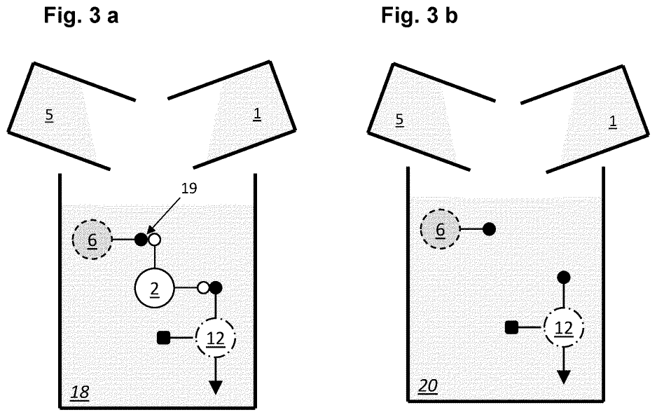

FIG. 3 a shows how the second fluid 18 is created in the case where the test substance particle 2 was originally present, in that the first nanobead fluid 5 and the test substance fluid 1 are mixed with the third nanobead fluid 11. The test substance particle 2 forms a binding of a first type 19 with the first magnetic nanobead 6 and the non-magnetic nanobead 12 in each case. FIG. 3 b shows the second fluid 20 in the case where the test substance particle was not present in the test fluid. Due to the test substance particle being absent, there is also no indirect connection between the second magnetic nanobead 6 and the nanobead 12.

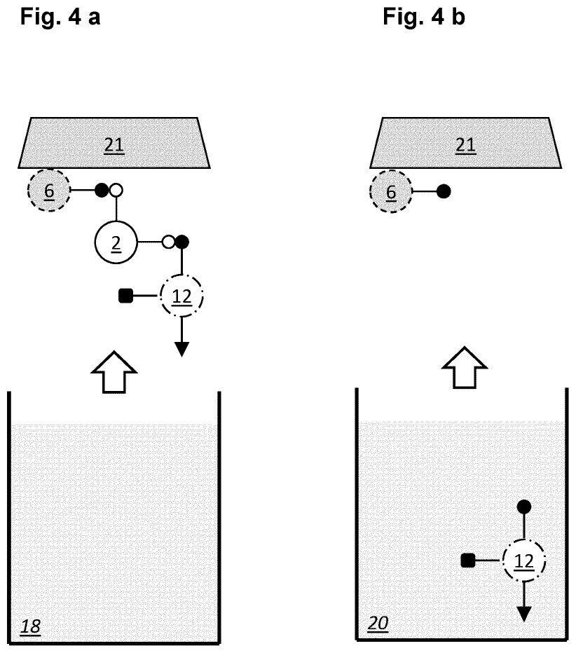

FIG. 4 a shows how the first magnetic nanobead 6 and the test substance particle 2 bound thereto and the non-magnetic nanobead 12 bound thereto are removed from the second fluid 18 with the first magnetic force generated by means of the first magnet 21 in the case where the test substance particle 2 was originally present. FIG. 4 b shows that, when the test substance particle 2 was originally absent, the non-magnetic nanobead remains in the second fluid because it is not indirectly bound to the first magnetic nanobead 6 via the test substance. In this case, however, the first magnetic nanobead 6 is removed from the second fluid by means of the first magnetic force applied by the first magnet 21, in the same way as when the test substance particle was originally present.

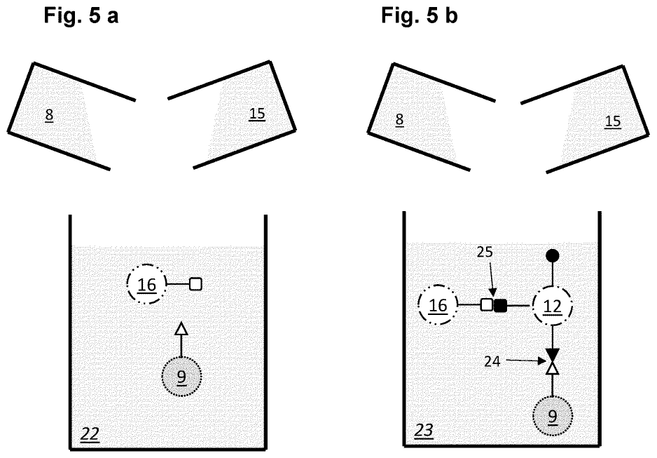

FIG. 5 a shows how the third fluid 22 is created in the case where the test substance particle was originally present, in that the second nanobead fluid 8 and the marker fluid 15 are mixed into the second fluid 18 from FIG. 4 a. Consequently, the second magnetic nanobead 9 and the marker particle 16 are present in the third fluid 22. FIG. 5 b shows the same process for the case where the test substance particle was originally absent. In this case, the non-magnetic nanobead 12 remained in the second fluid 20, as previously explained for FIG. 4 b. Said non-magnetic nanobead is now present in the third fluid 23 and forms a binding of a second type 24 with the second magnetic nanobead 9 and a binding of a third type 25 with the marker particle 16.

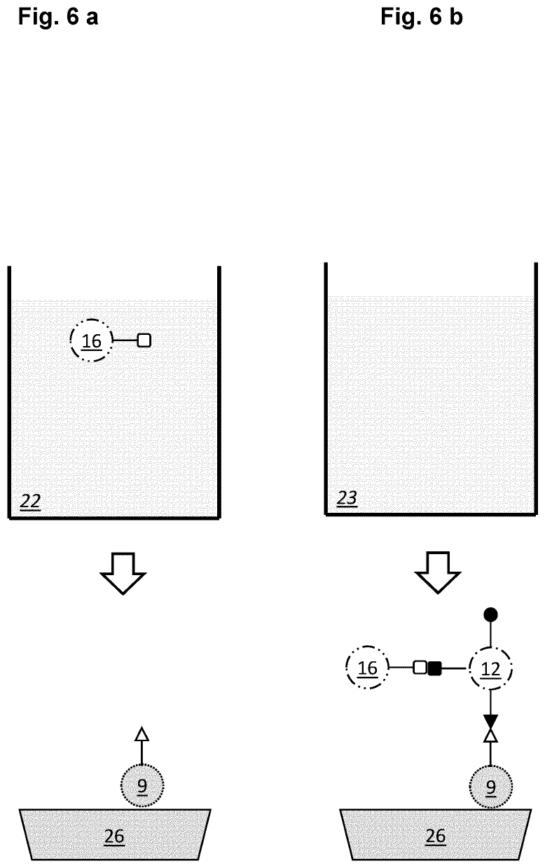

FIG. 6 a shows how the second magnetic nanobead 9 is removed from the third fluid 22 by means of the magnetic force generated by the second magnet 26 in the case where the test substance particle was originally present. The marker particle 16 remains in the third fluid 22. FIG. 6 b shows the process for the case where the test substance particle was originally absent. Due to the fact that, in this case, the non-magnetic nanobead 12 was present in the third fluid 23, the marker particle 16 is removed from the third fluid 23 by means of the magnetic force generated by the second magnet 26, because the marker particle 16 is indirectly bound to the second magnetic nanobead 9 via the non-magnetic nanobead 12.

When comparing the third fluid 22 in FIG. 6 a with the third fluid 23 in FIG. 6 b, it is apparent that the marker particle 16 only remains in the third fluid 22 in the case where the test substance particle was originally present. The marker particle alters the physical properties of the fluid 22 compared to the fluid 23, and therefore this difference can be used to easily ascertain whether the test substance particle was originally present. The alteration of the physical properties can be caused, for example, by fluorescence, which can be recorded with corresponding analytics.

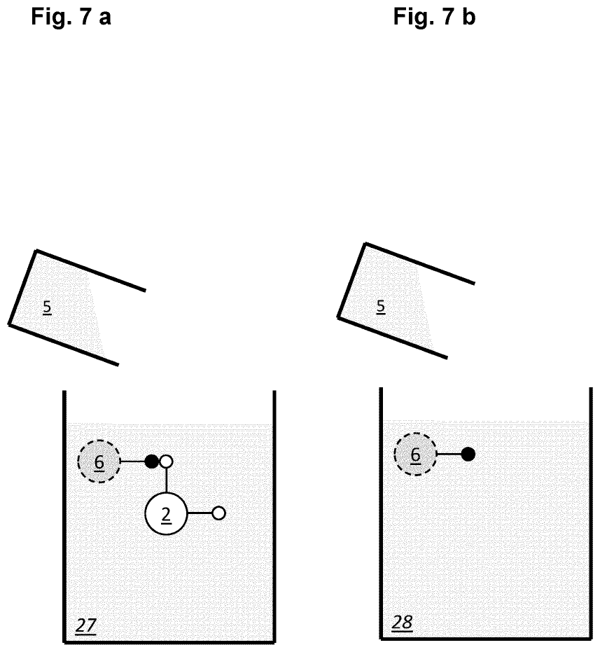

FIG. 7 a to 9 b show an alternative embodiment for creating the second fluid. The second fluid is not created in a single step, as shown in FIG. 3 a and FIG. 3 b, but rather in three substeps.

FIG. 7 a shows the first substep for the case where the test substance particle 2 was originally present in the test fluid. In this substep, the mixture 27 arises by mixing the first nanobead fluid 5 into the test fluid. Subsequently, the test substance particle 2 and the first magnetic nanobead 6 form a binding in the mixture 27. FIG. 7 shows the same substep for the case where the test substance particle was originally absent. Consequently, only the first magnetic nanobead 6 is found in the mixture 28 of the first nanobead fluid 5 and test fluid.

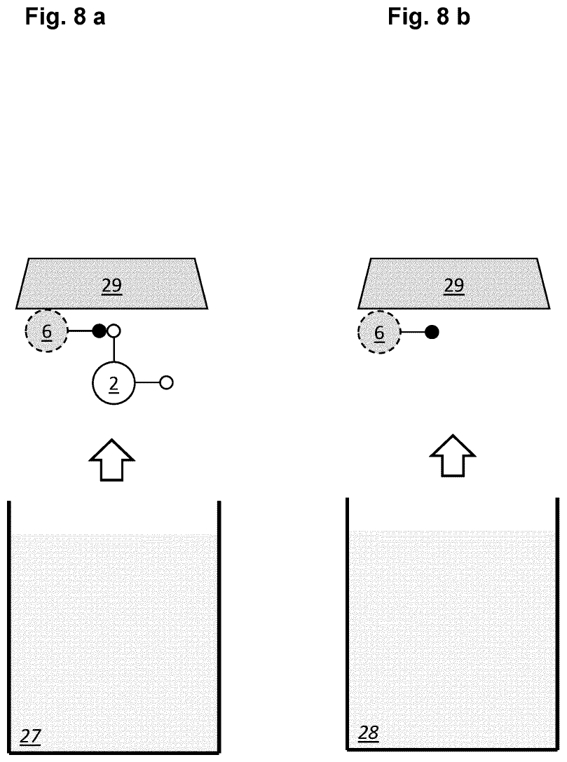

FIG. 8 a shows the next substep for the case where the test substance particle 2 was originally present. In this substep, the first magnetic nanobead 6 is removed from the mixture 27 together with the test substance particle 2 bound thereto by means of the magnetic force applied by the third magnet 29. FIG. 8 b shows the same step for the case where the test substance particle was originally absent. In this case, only the first magnetic nanobead is removed from the mixture 28.

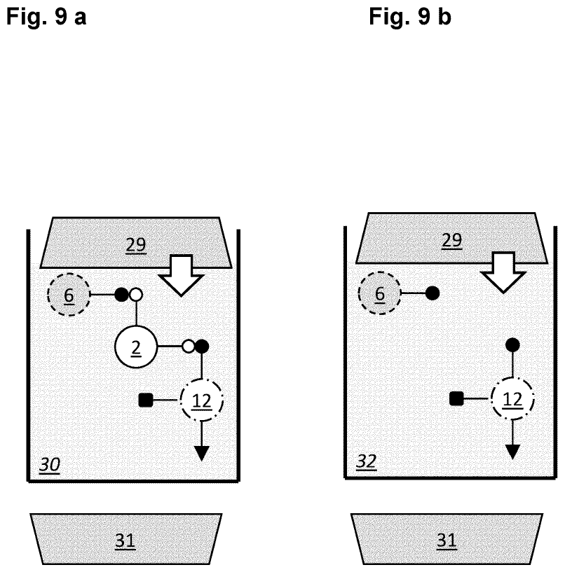

FIG. 9 a shows the next substep for the case where the test substance particle 2 was originally present. To create the second fluid 30, the test substance particle 2 is introduced into the third nanobead fluid 11 together with the first magnetic nanobead 6 by means of the fourth magnetic force generated by the fourth magnet 31. As a result, the third nanobead fluid 11 becomes the second fluid 30. To this end, the third magnet 29 is introduced into the third nanobead fluid 11 and then the fourth magnetic force is applied in that the fourth magnet 31 is held under the vessel of the third nanobead fluid 11. The fourth magnetic force applied by means of the fourth magnet 31 is greater than the third magnetic force, and therefore the second magnetic nanobead 6, including the test substance particle 2 bound thereto, held on the third magnet 29 is introduced into the third nanobead fluid 11. Since the third nanobead fluid 11 already contained the non-magnetic nanobead 12, the second fluid 30 thus also contains the non-magnetic nanobead 12. The test substance particle 2 and the non-magnetic nanobead 12 form a binding after being introduced into the third nanobead fluid, and therefore the first magnetic nanobead 6 is indirectly bound to the non-magnetic nanobead 12 via the test substance particle 2. FIG. 9 b shows the same substep for the case where the test substance particle was originally absent. In this case, no binding of the test substance particle to the first magnetic nanobead 6 took place, and therefore only the first magnetic nanobead 6 is introduced into the third nanobead fluid by means of the fourth magnetic force generated by the fourth magnet 31 in order to create the second fluid 32. Without the test substance, the first magnetic nanobead 6 cannot bind to the non-magnetic nanobead 12, not even indirectly.

The second fluid 30 from FIG. 9 a corresponds to the second fluid 18 from FIG. 3 a , while the second fluid 32 from FIG. 9 b corresponds to the second fluid 20 from FIG. 3 b.

On account of the alternative embodiment according to FIG. 7 a to FIG. 9 b, the non-magnetic nanobead 12 does not come into contact with the test fluid. This prevents so-called matrix effects from arising, which refer to an undesired reaction of the non-magnetic nanobead with impurities.

### LIST OF REFERENCE SIGNS

1 Test fluid with test substance particle present
2 Test substance particle
3 Binding site of a first type
4 Test fluid with test substance particle absent
5 First nanobead fluid
6 First magnetic nanobead
7 Complementary binding site of a first type
8 Second nanobead fluid
9 Second magnetic nanobead
10 Binding site of a second type
11 Third nanobead fluid
12 Non-magnetic nanobead
13 Complementary binding site of a second type
14 Complementary binding site of a third type
15 Marker fluid
16 Marker particle
17 Binding site of a third type
18 Second fluid with test substance particle present
19 Binding of a first type
20 Second fluid with test substance particle absent
21 First magnet
22 Third fluid with test substance particle present
23 Third fluid with test substance particle absent
24 Binding of a second type
25 Binding of a third type
26 Second magnet
27 Mixture of test substance fluid and first nanobead fluid with test substance particle present
28 Mixture of test substance fluid and first nanobead fluid with test substance particle absent
29 Third magnet
30 Second fluid with test substance particle present (method alternative)
31 Fourth magnet
32 Second fluid with test substance particle absent (method alternative)

1. A method for directly detecting the presence of a test substance in a test fluid, comprising:
providing a first fluid, the test fluid, in which a test substance particle is present in a first case and the test substance particle is not present in a second case,
wherein the test substance particle has binding sites of a first type,
providing
a first nanobead fluid containing a first magnetic nanobead with a complementary binding site of a first type,
a second nanobead fluid containing a second magnetic nanobead with a binding site of a second type,
a third nanobead fluid containing a non-magnetic nanobead with a complementary binding site of a first type, a complementary binding site of a second type, and a complementary binding site of a third type,
a marker fluid containing a marker particle with a binding site of a third type,
wherein, in the presence of corresponding binding partners in the same fluid,
a binding site of a first type of a test substance particle forms a binding of a first type with a complementary binding site of a first type of a magnetic nanobead of a first type and/or of a non-magnetic nanobead,
a binding site of a second type of a magnetic nanobead of a second type forms a binding of a second type with a complementary binding site of a second type of a non-magnetic nanobead,
a binding site of a third type of a marker particle forms a binding of a third type with a complementary binding site of a third type of a non-magnetic nanobead,
creating a second fluid
by mixing the test fluid with the first nanobead fluid and mixing at least the first magnetic nanobead of the resulting fluid with the third nanobead fluid, whereupon the resulting fluid is referred to as the second fluid,
removing the first magnetic nanobead from the second fluid by means of a first magnetic force,
wherein, in the first case, the test substance particle bound to the first magnetic nanobead and the non-magnetic nanobead bound to the test substance particle are also removed from the second fluid
or, in the second case, the non-magnetic nanobead remains in the second fluid,
creating a third fluid
by mixing the second nanobead fluid and the marker fluid into the second fluid, whereupon the mixture is referred to as the third fluid,
removing the second magnetic nanobead from the third fluid by means of a second magnetic force,
wherein, in the first case, due to the absence of the non-magnetic nanobead in the third fluid, the marker particle is not indirectly bound to the second magnetic nanobead, and therefore it remains in the third fluid after the second magnetic nanobead is removed, or
wherein, in the second case, the non-magnetic nanobead is bound to the second magnetic nanobead and the marker particle is bound to the non-magnetic nanobead, and therefore the marker particle is removed from the third fluid together with the second magnetic nanobead and the non-magnetic marker particle.

2. The method according to claim 1, wherein the third nanobead fluid is mixed with the test fluid and with the first nanobead fluid in order to create the second fluid.

3. The method according to claim 1, wherein, in order to create the second fluid,
firstly, the first nanobead fluid is mixed with the test fluid,
secondly, the first magnetic nanobead is removed from the test fluid by means of a third magnetic force, and
thirdly, the first magnetic nanobead removed from the test fluid is introduced into the third nanobead fluid by means of a fourth magnetic force.

4. The method according to claim 1, wherein
the test substance particle has two or more binding sites of a first type,
the first magnetic nanobead has a single or more complementary binding sites of a first type,
the second magnetic nanobead has a single or more binding sites of a second type,
the non-magnetic nanobead has a single or more complementary binding sites of a first type, a single or more complementary binding sites of a second type, a single or more complementary binding sites of a third type, and
the marker particle has a single or more binding sites of a third type.

5. The method according to claim 1, wherein
the test fluid contains a single or multiple test substance particles and/or
the first nanobead fluid contains a single or multiple first magnetic nanobeads and/or
the second nanobead fluid contains a single or multiple second magnetic nanobeads and/or
the third nanobead fluid contains a single or multiple non-magnetic nanobeads and/or
the first marker fluid contains a single or multiple marker particles.

6. The method according to claim 1, wherein the binding of a first type is an antibody-antigen binding or an oligonucleotide binding or a lipid binding or a glucose binding.

7. The method according to claim 1, wherein the binding of a second type is an antibody-antigen binding or an oligonucleotide binding or a lipid binding or a glucose binding.

8. The method according to claim 1, wherein the binding of a third type is an antibody-antigen binding or an oligonucleotide binding or a lipid binding or a glucose binding.

9. The method according to claim 1, wherein the marker particle is a colored particle, a luminous particle, a fluorescent particle, a particle that influences the electrical conductivity of a liquid, or a radioactive particle.

10. The method according to claim 1, wherein
an additional test substance particle with an alternative binding site of a first type is located in the test fluid,
an additional first nanobead fluid is provided which contains an alternative first nanobead with an alternative complementary binding site of a first type, and
an additional third nanobead fluid is provided which contains an alternative non-magnetic nanobead with an alternative complementary binding site of a first type and an alternative complementary binding site of a third type, and
an additional marker fluid is provided which contains an alternative marker particle with an alternative binding site of a third type,
the additional first nanobead fluid, the additional third nanobead fluid, and the additional marker fluid are used in the method in the same steps and in the same way as the first nanobead fluid, the third nanobead fluid, and the marker fluid, respectively.

11. The method according to claim 10, wherein
the test fluid contains a single or multiple additional test substance particles and/or
the first additional nanobead fluid contains a single or multiple alternative first nanobeads and/or
the third additional nanobead fluid contains a single or multiple alternative third nanobeads and/or
the additional marker fluid contains a single or multiple alternative marker particles.

12. The method according to claim 1, wherein
the first magnetic force is applied by means of a first magnet and/or
the second magnetic force is applied by means of a second magnet and/or
wherein
the relevant magnet is immersed in the relevant fluid,
as a result of magnetic forces, the relevant magnetic nanobead moves toward the relevant magnet and is then held on the relevant magnet,
such that the magnet and nanobead are removed together from the fluid in order to remove the nanobead.

13. The method according to claim 3, wherein, when creating the second fluid,
the third magnetic force is applied by means of a third magnet
which is immersed in the test fluid,
as a result of magnetic forces, the first magnetic nanobead moves toward the third magnet and is then held on the third magnet,
such that the magnet and first magnetic nanobead are removed together from the fluid in order to remove the first magnetic nanobead, and
the fourth magnetic force is generated by reversing the polarity of the third magnet, such that the first magnetic nanobead held on the magnet is repelled when the second fluid is created, wherein, in order to introduce the nanobead into the third nanobead fluid, the first magnet is first immersed in the third nanobead fluid before the polarity of the magnet is reversed.

14. The method according to claim 3, wherein, when creating the second fluid,
the third magnetic force is applied by means of a third magnet
which is immersed in the test fluid,
as a result of magnetic forces, the first magnetic nanobead moves toward the third magnet and is then held on the third magnet,
such that the magnet and first magnetic nanobead are removed together from the fluid in order to remove the first magnetic nanobead, and
the fourth magnetic force is generated by means of a magnetic field impressed on the third nanobead fluid from outside, wherein the fourth magnetic force is only impressed after the third magnet, with the first magnetic nanobead held on the third magnet, has been immersed in the third nanobead fluid.

15. The method according to claim 12, wherein the first and/or second and/or third magnet is surrounded by a protective layer, such that the relevant magnetic nanobead is held on the protective layer as a result of the magnetic forces.

16. The method according to claim 1, wherein the number of test substance particles originally present in the test fluid is determined in that
a known number of non-magnetic nanobeads, in each case with a known number of complementary binding sites of a first and third type, is used,
wherein the total number of complementary binding sites of a first type present on the non-magnetic nanobeads is greater than the number of test substance particles
and wherein the total number of complementary binding sites of a third type present on the non-magnetic nanobeads is less than the number of marker particles, and
the difference between the marker particles remaining in the third fluid at the end and the marker particles present at the start is determined,
the number of test substances originally present is calculated using the difference of the marker particles with knowledge of the number of non-magnetic

17. The method according to claim 1, wherein the container in which the second fluid is created tapers conically downward.

18. The method according to claim 1, which is used to detect the test substance in the test fluid if the number of test substance particles is within the femto- and/or atto- and/or zeptomolar range.

19. The method according to claim 1, which is used for single-molecule counting.
